(19) United States
(12) Patent Application Publication
(10) Pub. No.: US 2022/0347655 A1
(43) Pub. Date: Nov. 3, 2022

(54) WATER-ABSORBENT CROSS-LINKED POLYMERIC POLYCARBOXYLIC ACID AND METHODS OF MAKING

(71) Applicant: Ecovia Renewables Inc., Ann Arbor, MI (US)

(72) Inventor: Peng Chao, Ann Arbor, MI (US)

(73) Assignee: Ecovia Renewables Inc., Ann Arbor, MI (US)

(21) Appl. No.: 17/765,867

(22) PCT Filed: Oct. 2, 2020
(86) PCT No.: PCT/US20/53938

§ 371 Date: Apr. 1, 2022

Related U.S. Application Data
(60) Provisional application No. 62/910,648, filed on Oct. 4, 2019.

(51) Int. Cl. B01J 20/26; C08G 69/48; C08G 69/08; B01J 20/30

(52) U.S. Cl. CPC B01J 20/267; C08G 69/48; C08G 69/08; B01J 20/3085

(57) ABSTRACT

Disclosed are methods of preparing a cross-linked polymeric polycarboxylic acid by cross-linking the polymeric polycarboxylic acid with cross-linking agent comprising a polyepoxide and a polyhydrazide. Also disclosed are the cross-linked polymeric polycarboxylic acids made by the process and water-absorbent polymer materials comprising the cross-linked polymeric polycarboxylic acids.

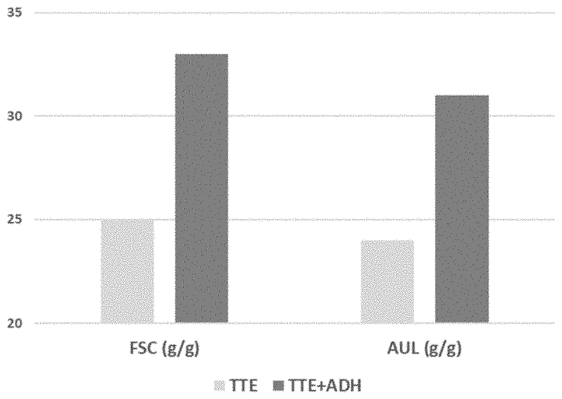

This application claims the benefit of U.S. Provisional Application No. 62/910,648, filed Oct. 4, 2019, which is hereby incorporated herein by reference.

This invention was made with government support under IIP 1660217 awarded by the National Science Foundation. The government has certain rights in this invention.

### FIELD OF THE INVENTION

The disclosed invention relates to methods of preparing cross-linked polymeric polycarboxylic acids and water-absorbent polymer materials.

### INTRODUCTION

Water-absorbing polymers, which absorb water or aqueous fluid and hold it in the form of a gel, have been used in many applications, such as for hygiene products like disposable diapers, agricultural products like soil amendments, and other applications in which absorption, retention, or delivery of water is useful.

Traditionally such water-absorbent materials have been made of synthetic petroleum-based polymers such as the sodium salt of poly(acrylic acid) and polyacrylamide that are cross-linked into water insoluble networks that can absorb water to form hydrous gels. Although relatively inexpensive, petroleum-based polymers have a negative impact on the environment due, among other things, to their non-renewable and non-degradable natures and regulated emissions generated from the processes for obtaining their constituent monomers from petroleum sources.

A renewable alternative to such traditional absorbent polymer materials is absorbent material using bio-based polymers, such as poly(amino acids) and polysaccharides. For example, gamma-poly(glutamic acid) (γ-PGA) is a water-soluble polymeric polycarboxylic acid that can be commercially manufactured by a microbial fermentation process. γ-PGA has a hydrophilic polyamide backbone and, like poly(acrylic acid), has a pendent carboxylic acid functional group in each repeating unit. These features make it suitable for cross-linking into a material for use in absorbent applications. For example, γ-PGA can be cross-linked by glycidyl ether cross-linkers such as ethylene glycol diglycidyl ether and trimethylolpropane triglycidyl ether to form a water-absorbing product. However, the absorbency of the glycidyl ether-cross-linked γ-PGA is not ideal compared to the traditional cross-linked sodium polyacrylate due to lower free swell capacity (FSC) and absorbency under load (AUL), which limit their applications.

### SUMMARY OF THE DISCLOSURE

Disclosed are methods of preparing a cross-linked polymeric polycarboxylic acid by cross-linking the polymeric polycarboxylic acid with a cross-linking agent comprising a polyepoxide and a polyhydrazide. Also disclosed are the cross-linked polymeric polycarboxylic acids made by the methods, water-absorbent cross-linked polymeric polycarboxylic acids made by the methods, and water-absorbent materials comprising the cross-linked polymeric polycarboxylic acids.

### BRIEF DESCRIPTION OF THE DRAWINGS

The FIGURE shows the free swell capacity (FSC) and absorbency under load (AUL) of a cross-linked γ-PGA embodiment of the invention.

### DETAILED DESCRIPTION

“A,” “an,” “the,” “at least one,” and “one or more” are used interchangeably to indicate that at least one of the item is present; a plurality of such items may be present unless the context clearly indicates otherwise. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. In addition, disclosure of ranges includes disclosure of all values and further divided ranges within the entire range. The terms “comprises,” “comprising,” “including,” and “having,” are inclusive and therefore specify the presence of stated features, integers, steps, operations, elements, or components, but do not preclude the presence or addition of one or more other features, integers, steps, operations, elements, components, or groups thereof. As used in this specification, the term “or” includes any and all combinations of one or more of the associated listed items. A “water-soluble” polymer is a polymer that can be combined with water, with or without the presence of co-solvents and/or neutralizing agents, to form transparent solutions. A “water-dispersible” polymer is a polymer that can be combined with water, with or without the presence of co-solvents and/or neutralizing agents, to form a stable dispersion. A dispersion that has no visible settled sedimentation after 24 hours storage at 25° C. may be considered to be stable.

A cross-linked polymeric polycarboxylic acid is prepared by cross-linking a polymeric polycarboxylic acid with a polyepoxide compound and a polyhydrazide compound. The polymeric polycarboxylic acid is a polymer having carboxylic acid groups pendent along the polymer backbone. The polymeric polycarboxylic acid may optionally have a carboxylic acid group on one or both ends of the polymer chain. In various embodiments, the polymeric polycarboxylic acid may have a carboxylic acid group pendent from every monomer unit to, on average, a carboxylic acid group pendent from about every tenth monomer unit; or pendent from every monomer unit to, on average, pendent from about every sixth monomer unit; or pendent from every monomer unit to, on average, pendent from about every fifth monomer unit; or pendent from every monomer unit to, on average, pendent from about every fourth monomer unit; or pendent from every monomer unit to, on average, pendent from about every third monomer unit; or pendent from every monomer unit to, on average, pendent from about every other monomer unit; or pendent from every monomer unit.

The weight-average molecular weight of the polymeric polycarboxylic acid may be from about 1 kDa to about 50,000 kDa, preferably from about 5 kDa to about 50,000 kDa, more preferably from about 100 kDa to about 5,000 kDa, still more preferably from about 200 kDa to about 600 kDa, determined by gel permeation chromatography (GPC) equipped with a light scattering detector. In various embodiments, the weight-average molecular weight of the polymeric polycarboxylic acid may be from about 1 kDa or from about 5 kDa or from about 10 kDa or from about 20 kDa or from about 30 kDa or from about 50 kDa or from about 100 kDa or from about 150 kDa or from about 200 kDa or from about 250 kDa or from about 300 kDa up to about 500 kDa or up to about 550 kDa or up to about 600 kDa or up to about 700 kDa or up to about 800 kDa or up to about 900 kDa or up to about 1000 kDa or up to about 2000 kDa or up to about 5000 kDa or up to about 7500 kDa or up to about 10,000 kDa or up to about 15,000 kDa or up to about 20,000 kDa or up to about 25,000 kDa or up to about 30,000 kDa or up to about 40,000 kDa or or up to about 50,000 kDa. The polymeric polycarboxylic acid preferably contains a sufficient number of carboxylic acid groups to be water-soluble or water-dispersible. In certain embodiments, the average number of carboxylic acid groups per polymeric polycarboxylic acid chain may be from about 2 to about 700,000, preferably from about 50 to about 50,000, and more preferably from about 1,500 to about 8,000.

Nonlimiting examples of suitable polycarboxylic acid polymers for cross-linking include homopolymers and copolymers of ethylenically unsaturated carboxylic acids such as acrylic acid, methacrylic acid, 2-ethacrylic acid, 2-propylacrylic acid, crotonic acid, itaconic acid, maleic acid, fumaric acid, and the like, as well as salts and anhydrides of these; carboxymethyl cellulose and salts thereof polyaspartic acids and salts thereof; polyglutamic acids and salts thereof and carboxyethyl dextran and salts thereof. In certain embodiments, the polycarboxylic acid polymer may be selected from the group consisting of α-poly(glutamic acid), γ-poly(glutamic acid), α-poly(aspartic acid), ß-poly(aspartic acid), carboxymethyl cellulose, poly(acrylic acid), poly(methacrylic acid), poly(2-carboxyethyl acrylate), poly(2-ethylacrylic acid), poly(2-propylacrylic acid), poly(maleic acid), their copolymers, and combinations thereof. In certain embodiments, the polymeric polycarboxylic acid is or includes a poly(amino acid), for example a homopolymer of aspartic or glutamic acid such as L-α-poly(aspartate) or L-α-poly(glutamate) or combinations thereof produced through a ribosomal translation method. Other nonlimiting examples of useful poly(amino acids) include D,L-(α,β)-poly(aspartate) or D,L-(α, γ)-poly(glutamate) or combinations thereof produced from aspartic acid and/or glutamic acid monomers through condensation polymerization or D-γ-poly(glutamate), L-γ-poly(glutamate), D,L-γ-poly(glutamate) or any combination of these produced through non-ribosomal synthesis in a microbial fermentation or in vitro biochemical method. The polycarboxylic acid polymer may be used in any combination in the cross-linking process.

In addition to the polymeric polycarboxylic acid or combination of polymeric polycarboxylic acids, the reaction may further comprise a second polymer having a plurality of groups reactive with the cross-linking agent, for example a plurality of reactive groups selected from the group consisting of carboxylic acid groups, amine groups, hydroxyl groups, and combinations thereof. In various embodiments, the second polymer is water soluble or water dispersible. Nonlimiting examples of polymers suitable as the second polymer include starch, guar gum, xanthan gum, carrageenan, pectin, glucomannan, inulin, cellulose, β-glucan, dextrin, galactomannan, alginic acid, chitosan, homopolymers and copolymers of ethylenically unsaturated carboxylic acids, amines, and alcohols, such as acrylic acid, methacrylic acid, 2-ethacrylic acid, 2-propylacrylic acid, acrylamide, 2-hydroxyethyl acrylate, N-(2-hydroxyethyl) acrylamide, maleic acid, and 2-aminoethyl methacrylate, and combinations of such polymers.

The polymeric polycarboxylic acid or a combination of polymeric polycarboxylic acids, is cross-linked by reaction with a cross-linking agent comprising a polyepoxide and a polyhydrazide. This cross-linking agent is found to increase water absorbency of the product when a water-soluble or water-dispersible polymeric polycarboxylic acid is crosslinked. In particular, both free-swell capacity and absorbency under load are increased using the cross-linking agent comprising a polyepoxide and a polyhydrazide as compared to using a polyepoxide crosslinking agent alone. To determine the free swell capacity, a tea bag containing 0.1 g of the crosslinked polymeric polycarboxylic acid is soaked in 100 mL of 0.9% NaCl solution for 5 minutes at room temperature (23±2° C.). Then, the tea bag is removed from the saline solution and hung for 5 minutes to remove the water on the surface. The swollen cross-linked product is then weighed. The free swell capacity is defined as the ratio of the weight of the water absorbed (the difference between the wet and dry weights) to the dry weight. To determine the absorbency under load, 0.1 g of the crosslinked polymeric polycarboxylic acid is placed into a plastic cylinder having a screen fabric on the bottom and a plastic piston was placed on the product (0.3 psi). A filter sponge is placed in a glass container and the container is filled with 0.9% NaCl solution up to the edge of the filter sponge. Then, the assembly containing the product is placed on the filter sponge for 90 minutes at room temperature (23±2° C.). The absorbency under load is calculated by the ratio of the weight of the water absorbed (the difference between the wet and dry weights) to the dry weight.

Suitable polyepoxide cross-linking molecules contain two or more reactive epoxide groups. Nonlimiting examples of these include, but are not limited to, polyglycidyl ethers of alkanepolyols and poly(alkylene glycols), including, for further example, ethylene glycol diglycidyl ether, diethylene glycol diglycidyl ether, polyethylene glycol diglycidyl ether, glycerine diglycidyl ether and triglycidyl ether, propylene glycol diglycidyl ether, butanediol diglycidyl ether, and polyglycidyl ethers of erythritol, trimethylolethane, pentaerythritol, and trimethyolpropane; diepoxyalkanes and diepoxyaralkanes, including, 1,2,3,4-diepoxybutane, 1,2,4,5-diepoxypentane, 1,2,5,6-diepoxyhexane, 1,2,7,8-diepoxyoctane, 1,4- and 1,3-divinylbenzene diepoxides; polyphenol polyglycidyl ethers, including, for further example, 4,4′-isopropylidenediphenol diglycidyl ether (bisphenol A diglycidyl ether) and hydroquinone diglycidyl ether; and polyglycidyl esters of polycarboxylic acids such as oxalic acid diglycidyl ester, succinic acid diglycidyl ester, adipic acid diglycidyl ester, glutaric acid diglycidyl ester, phthalic acid diglycidyl ester, terephthalic acid diglycidyl ester, hexahydrophthalic acid diglycidyl ester, 2,6-napthalenedicarboxylic acid diglycidyl ester, as well as epoxide esters of polyunsaturated fatty acids and their oliogomers, such polyepoxidized dimerized linolenic acid, polyepoxidized linoleic acids, polyepoxidized linolenic acids including the polyepoxided derivatives of linseed oil, soybean oil, alkyl esters of these, and oligomers of these.

In certain embodiments, the cross-linking agent comprises a polyepoxide selected from the group consisting of polyepoxides having a structure as shown in Formula (I):

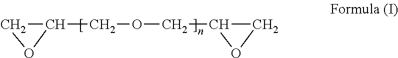

wherein n is from 1 to 150;
and polyepoxides having a structure as shown in Formula (II)

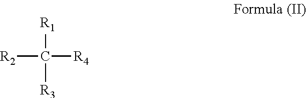

wherein R₁ is H, CH₃, CH₂CH₃, OH, CH₂OH,

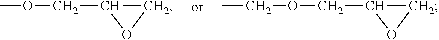

R₂, R₃, and R₄ is

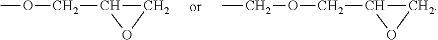

The polyepoxide may be a member selected from the group consisting of ethylene glycol diglycidyl ether, diethylene glycol diglycidyl ether, propylene glycol diglycidyl ether, 1,3-butanediol diglycidyl ether, bisphenol A diglycidyl ether, bisphenol F diglycidyl ether, 1,4-butanediol diglycidyl ether, neopentyl glycol diglycidyl ether, resorcinol diglycidyl ether, glycerol diglycidyl ether, glycerol triglycidyl ether, poly(ethylene glycol) diglycidyl ethers, poly(propylene glycol) diglycidyl ethers, trimethylolpropane diglycidyl ether, trimethylolethane triglycidyl ether, triethylolpropane diglycidyl ether, triethylolethane triglycidyl ether, glycerol propoxylate triglycidyl ether, pentaerythritol tetraglycidyl ether, castor oil polyglycidyl ether, sorbitol polyglycidyl ether, polyglycerol polyglycidyl ether, and combinations thereof.

The cross-linking agent further comprises a polyhydrazide having at least two hydrazide functional groups. Nonlimiting examples of suitable polyhydrazide include polyhydrazides of aliphatic and aromatic dicarboxylic acids and tricarboxylic acid, such as adipic acid dihydrazide, citric acid dihydrazide and trihydrazide, oxalic acid dihydrazide, succinic acid dihydrazide, glutaric acid dihydrazide, phthalic acid dihydrazide, terephthalic aciddihydrazide, hexahydrophthalic acid dihydrazide, and 2,6-napthalenedicarboxylic acid dihydrazide.

In certain embodiments, the cross-linking agent comprises a polyhydrazide selected from the group consisting of polyhydrazides having a structure as shown in Formula (III):

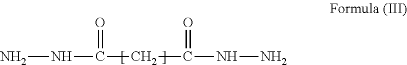

wherein n is from 1 to 10;
and polyhydrazides having a structure as shown in Formula (IV)

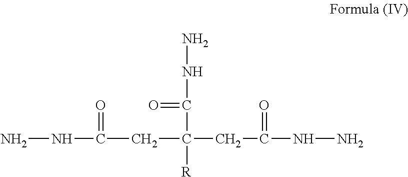

wherein R is H, OH, or CH₃.

The polyhydrazide may be a member selected from the group consisting of oxalyl dihydrazide, succinic acid dihydrazide, malonic acid dihydrazide, ethylmalonic acid dihydrazide, adipic acid dihydrazide, pimelic acid dihydrazide, dodecanedioic dihydrazide, sebacic acid dihydrazide, phthalic acid dihydrazide, terephthalic acid dihydrazide, isophthalic acid dihydrazide, carbohydrazide, thiocarbohydrazide, citric acid trihydrazide, ethylenediaminetetraacetic acid tetrahydrazide, and combinations thereof.

The cross-linking agent may comprise a plurality of polyepoxides or a plurality of polyhydrazides, or a plurality of polyhydrazides and a plurality of polyepoxides. The cross-linking agent may comprise a further reactant in a minor amount, such as up to about 10 mol % based on total moles of cross-linking compounds. Examples of such further reactants include compounds with a plurality of aziridine groups, carbodiimide compounds, amine compounds with at least two active hydrogens, and monomeric polycarboxylic acids.

In certain embodiments, the amount of polyepoxide and the amount of polyhydrazide used in the cross-linking reaction are each independently preferably from about 0.1 to about 10 wt %, more preferably from about 0.5 to about 5 wt %, and even more preferably from about 1 to about 3 wt % based on the weight of the polymeric polycarboxylic acid. In certain embodiments, the amount of polyepoxide and the amount of polyhydrazide used in the cross-linking reaction are each independently preferably from about 0.1 to about 10 mol %, more preferably from about 0.5 to about 5 mol %, and even more preferably from about 1 to about 3 mol % based on the acid equivalent weight of the polymeric polycarboxylic acid. In certain embodiments, the molar ratio between the polyepoxide and the polyhydrazide is preferably from about 0.1 to about 10, more preferably from about 0.2 to about 5, and even more preferably from about 0.5 to about 2.

In an example embodiment, all the components for the cross-linking reaction are dissolved in an aqueous medium and the reaction solution is heated in an oven. The concentration of the polymeric polycarboxylic acid in the reaction solution may be from about 10 to about 300 g/L, preferably from about 50 to about 200 g/L, and more preferably from about 80 to about 150 g/L. The pH of the reaction solution may be from about 3 to about 9, preferably from about 4 to about 8, and more preferably from about 5 to about 7. Useful neutralizing agents include alkali metal bases, ammonia, and, or amines. The oven temperature may be from about 50 to about 200° C., preferably from about 80 to about 180° C., and more preferably from about 100 to about 150° C. The reaction mixture may be kept in the oven from about 1 to about 12 hours, preferably from about 1.2 to about 6 hours, and more preferably from about 1.5 to about 3 hours.

If desired, drying may be carried out in an oven, such as a forced air oven, at any of the oven temperatures given above, or with infrared heating at a temperature from about 20 to about 180° C.

The cross-linking reaction may be carried out in an aqueous medium. The crosslinked polymeric polycarboxylic acid product may then be dried, pulverized, and classified to provide a particulate crosslinked polymeric polycarboxylic acid of a desired average particle size and/or particle size distribution. Nonlimiting examples of pulverizers include vertical pulverizers, grinders, rotary cutter mills, disc mills, and other such cutting, grinding, or crushing devices. In an example, the cross-linked polymeric polycarboxylic acid may be further dried after a coarse pulverization, then ground or crushed, for example in a suitable mill, and classified to a final desired average particle size.

The pulverized, crosslinked polymeric polycarboxylic acid is not limited to any particular particle shape or geometry. The particulate crosslinked polymeric polycarboxylic acid may be in the form of a powder, flakes, agglomerates, granules, irregular granular particles, spheres, ellipsoids, cylindrically-shaped particles (or whiskers), fibers, or another shape suitable for its intended use. Example uses include, without limitation, in baby diapers and adult hygiene products, as soil additives, for oil treatment and industrial dewatering, for medical applications such as devices for drug delivery and implants for tissue engineering, as a thickener for aqueous media including for personal care and food products, and other applications requiring absorption, desorption, or thickening of water or aqueous fluid.

In some embodiments, the composition of particles of cross-linked polymeric polycarboxylic acid further comprises excipients or additives that enhance performance or ease of use in end applications. The type of excipient or additive is not particularly limited. Suitable examples include, but are not limited to, other molecular species that are cross-linked with the polymeric polycarboxylic acid to alter material properties, surfactants or emulsifiers to enhance dispersion, inorganic fillers to enhance mechanical properties, coating the particles of cross-linked polymeric polycarboxylic acid with an active formulation ingredient, or impregnating the particles of cross-linked polymeric polycarboxylic acid with an active formulation ingredient.

Testing shows the cross-linked polymeric polycarboxylic acid exhibits much higher water absorbency when polyepoxide and polyhydrazide were both used for the cross-linking reaction compared to the one that used only polyepoxide as the cross-linker. In addition, the use of polyhydrazide as the only cross-linker did not provide a cross-linked product. Without wishing to be bound to a particular theory, it is believed that the improved properties are due to reaction between and polyepoxide and polyhydrazide that forms a unique linkage between the polymer chains of the cross-linked polycarboxlic acid.

This invention will be further described by the following examples. It should be noted that the working examples are provided for an illustration of the present invention, rather than intended to limit the scope of the present invention.

### Examples

The weight-average molecular weight of the γ-PGA used in the examples is 255 kDa, as determined by gel permeation chromatography equipped with a light scattering detector. The γ-PGA (10 g) was dispersed in DI water (100 mL) with an immersion blender and the pH of the solution was adjusted to 5.5 by adding 4 M HCl (100 μL). Then, trimethylolpropane triglycidyl ether (TTE) (200 μL) and adipic acid dihydrazide (ADH) (100 mg) were added. The mixture was poured onto a silicone mat and heated at 150° C. for 2 hours. After that, the product was purified by soaking in a large volume of DI water overnight, dried in a dehydrator at 45° C. for 48 h, and then ground to particles (20-100 mesh). The cross-linked product was then tested for its water absorbency, including free swell capacity (FSC) and absorbency under load (AUL).

For comparison, the cross-linking of γ-PGA was also attempted using trimethylolpropane triglycidyl ether under the same condition. The linear γ-PGA (10 g) was dispersed in DI water (100 mL) with an immersion blender and the pH of the solution was adjusted to 5.5 by adding 4 M HCl (100 μL). Then, trimethylolpropane triglycidyl ether (TTE) (200 μL) was added. The mixture was poured onto a silicone mat and heated at 150° C. for 2 hours. After that, the product was purified by soaking in a large volume of DI water overnight, dried in a dehydrator at 45° C. for 48 h, and then ground to particles (20-100 mesh).

For comparison, the cross-linking of γ-PGA was also attempted using adipic acid dihydrazide under the same condition. The linear γ-PGA (10 g) was dispersed in DI water (100 mL) with an immersion blender and the pH of the solution was adjusted to 5.5 by adding 4 M HCl (100 μL). Then, adipic acid dihydrazide (ADH) (100 mg) was added. The mixture was poured onto a silicone mat and heated at 150° C. for 2 hours. However, the preparation of adipic acid dihydrazide cross-linked γ-PGA was not successful. The obtained product was water-soluble and did not form a gel.

To determine the free swell capacity (FSC), a tea bag containing 0.1 g of the product was soaked in 100 mL of 0.9% NaCl solution for 5 minutes at room temperature (23±2° C.). Then, the tea bag was removed from the saline solution and was hung diagonally for 5 minutes to remove excess saline solution by dripping. After that, the swollen cross-linked product was weighed. The free swell capacity is calculated by the ratio of the weight of the water absorbed (the difference between the wet and dry weights) to the dry weight. The free swell capacity of the examples are shown in the FIGURE.

To determine the absorbency under load (AUL), 0.1 g of the product was placed into a plastic cylinder having a screen fabric on the bottom and a plastic piston was placed on the product (0.3 psi). A filter sponge was placed in a glass container and the container was filled with 0.9% NaCl solution up to the edge of the filter sponge. Then, the assembly containing the product was placed on the filter sponge for 90 minutes at room temperature (23±2° C.). The absorbency under load is calculated by the ratio of the weight of the water absorbed (the difference between the wet and dry weights) to the dry weight. The absorbency under load of the examples are shown in the FIGURE.

As shown in the FIGURE, the γ-PGA cross-linked by TTE/ADH showed much higher FSC (33 g/g vs. 25 g/g) and AUL (31 g/g vs. 24 g/g) compared to the γ-PGA cross-linked by TTE, which is 32% improvement for FSC and 29% improvement for AUL.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

What is claimed is:

1. A method of preparing a water-absorbent cross-linked polymeric polycarboxylic acid comprising cross-linking the polymeric polycarboxylic acid with a cross-linking agent comprising a polyepoxide and a polyhydrazide.

2. The method according to claim 1, wherein the cross-linking agent consists of a member selected from the group consisting of polyepoxides and combinations thereof and a member selected from the group consisting of polyhydrazides and combinations thereof.

3. The method according to claim 1, wherein the polymeric polycarboxylic acid has a carboxylic acid group pendent from every monomer unit to, on average, a carboxylic acid group pendent from about every tenth monomer unit.

4. The method according to claim 1, wherein the polymeric polycarboxylic acid has a weight-average molecular weight of from about 1 kDa to about 50,000 kDa.

5. The method according to claim 3, wherein the polymeric polycarboxylic acid contains a sufficient number of carboxylic acid groups to be water-soluble or water-dispersible.

6. The method according to claim 5, wherein the polymeric polycarboxylic acid has an average number of carboxylic acid groups per polymeric polycarboxylic acid chain of from about 2 to about 700,000.

7. The method according to claim 3, wherein the polymeric polycarboxylic acid comprises a member selected from the group consisting of homopolymers and copolymers of ethylenically unsaturated carboxylic acids and salts and anhydrides thereof; carboxymethyl cellulose and salts thereof; polyaspartic acids and salts thereof; polyglutamic acids and salts thereof; and carboxylethyl dextran and salts thereof.

8. The method according to claim 7, wherein the polymeric polycarboxylic acid comprises a member selected from the group consisting of α-poly(glutamic acid), γ-poly(glutamic acid), α-poly(aspartic acid), ß-poly(aspartic acid), carboxymethyl cellulose, poly(acrylic acid), poly(methacrylic acid), poly(2-carboxyethyl acrylate), poly(2-ethylacrylic acid), poly(2-propyl acrylic acid), poly(maleic acid), their copolymers, and combinations thereof.

9. The method according to claim 8, wherein the polymeric polycarboxylic acid comprises γ-poly(glutamic acid).

10. The method according claim 8, wherein the polymeric polycarboxylic acid is water-soluble or water-dispersible.

11. The method according to claim 3, wherein a second polymer having a plurality of groups reactive with the cross-linking agent is cross-linked with the polymeric polycarboxylic acid.

12. The method according to claim 3, wherein the polyepoxide is a member selected from the group consisting of ethylene glycol diglycidyl ether, diethylene glycol diglycidyl ether, propylene glycol diglycidyl ether, 1,3-butanediol diglycidyl ether, bisphenol A diglycidyl ether, bisphenol F diglycidyl ether, 1,4-butanediol diglycidyl ether, neopentyl glycol diglycidyl ether, resorcinol diglycidyl ether, glycerol diglycidyl ether, glycerol tiiglycidyl ether, poly(ethylene glycol) diglycidyl ethers, poly(propylene glycol) diglycidyl ethers, trimethylolpropane diglycidyl ether, trimethylolethane triglycidyl ether, triethylolpropane diglycidyl ether, triethylolethane triglycidyl ether, glycerol propoxylate triglycidyl ether, pentaerythritol tetraglycidyl ether, castor oil polyglycidyl ether, sorbitol polyglycidyl ether, polyglycerol polyglycidyl ether, and combinations thereof, and
the polyhydrazide is a member selected from the group consisting of oxalyl dihydrazide, succinic acid dihydrazide, malonic acid dihydrazide, ethylmalonic acid dihydrazide, adipic acid dihydrazide, pimelic acid dihydrazide, dodecanedioic dihydrazide, sebacic acid dihydrazide, phthalic acid dihydrazide, terephthalic acid dihydrazide, isophthalic acid dihydrazide, carbohydrazide, thiocarbohydrazide, citric acid trihydrazide, ethylenediaminetetraacetic acid tetrahydrazide, and combinations thereof.

13. (canceled)

14. The method according to claim 12, wherein the cross-linking agent comprises from about 0.1 to about 10 wt % of the polyepoxide and/or from about 0.1 to about 10 wt % of the polyhydrazide based on the weight of the polymeric polycarboxylic acid.

15. The method according to claim 12, wherein the molar ratio between the polyepoxide and the polyhydrazide is from about 0.1 to about 10.

16. The water-absorbent cross-linked polymeric polycarboxylic acid made by the method of claim 12.

17. An absorbent material comprising the water-absorbent cross-linked polymeric polycarboxylic acid according to claim 16.

18. The method according to claim 1, wherein the polymeric polycarboxylic acid comprises a member selected from the group consisting of α-poly(glutamic acid), γ-poly(glutamic acid), α-poly(aspartic acid), ß-poly(aspartic acid), carboxymethyl cellulose, poly(acrylic acid), poly(methacrylic acid), poly(2-carboxyethyl acrylate), poly(2-ethylacrylic acid), poly(2-propylacrylic acid), poly(maleic acid), their copolymers, and combinations thereof.

19. The method according to claim 18, wherein the cross-linking agent comprises a polyepoxide and a polyhydrazide, wherein the polyepoxide is a member selected from the group consisting of ethylene glycol diglycidyl ether, diethylene glycol diglycidyl ether, propylene glycol diglycidyl ether, 1,3-butanediol diglycidyl ether, bisphenol A diglycidyl ether, bisphenol F diglycidyl ether, 1,4-butanediol diglycidyl ether, neopentyl glycol diglycidyl ether, resorcinol diglycidyl ether, glycerol diglycidyl ether, glycerol tiiglycidyl ether, poly(ethylene glycol) diglycidyl ethers, poly(propylene glycol) diglycidyl ethers, trimethylolpropane diglycidyl ether, trimethylolethane triglycidyl ether, triethylolpropane diglycidyl ether, triethylolethane triglycidyl ether, glycerol propoxylate triglycidyl ether, pentaerythritol tetraglycidyl ether, castor oil polyglycidyl ether, sorbitol polyglycidyl ether, polyglycerol polyglycidyl ether, and combinations thereof, and
the polyhydrazide is a member selected from the group consisting of oxalyl dihydrazide, succinic acid dihydrazide, malonic acid dihydrazide, ethylmalonic acid dihydrazide, adipic acid dihydrazide, pimelic acid dihydrazide, dodecanedioic dihydrazide, sebacic acid dihydrazide, phthalic acid dihydrazide, terephthalic acid dihydrazide, isophthalic acid dihydrazide, carbohydrazide, thiocarbohydrazide, citric acid trihydrazide, ethylenediaminetetraacetic acid tetrahydrazide, and combinations thereof.

20. The method according to claim 18, wherein the polymeric polycarboxylic acid is water-soluble or water-dispersible.

21. A cross-linked polymeric polycarboxylic acid made by the method of claim 20, wherein the cross-linked polymeric polycarboxylic acid is water-soluble or water-dispersible.
